(19) United States
(12) Patent Application Publication
(10) Pub. No.: US 2002/0164780 A1
(43) Pub. Date: Nov. 7, 2002

(54) Micro organism cultivation device

(76) Inventors: Nan-Kuang Yao, Hsinchu (TW); Kuo-Yao Weng, Hsinchu (TW)

Correspondence Address: BACON & THOMAS, PLLC, 4th Floor, 625 Slaters Lane, Alexandria, VA, 22314-1176, US

(21) Appl. No.: 09/796,623

(22) Filed: Mar. 2, 2001

(51) Int. Cl. C12M 3/00; C12N 13/00

(52) U.S. Cl. 435/289.1; 435/293.1; 435/305.2; 435/173.8

(57) ABSTRACT

In the micro organism cultivation device of this invention, an implantable bio-artificial micro device, i.e., a cell apartment, is provided to cultivate cells or tissues. The cells or tissues to be cultivated include that secrete hormones such as islets of Langerhans. At both sides of the cell apartment, provided are microfluidic channels comprising dynamic micro-electric field array filters. The dynamic micro-electric field array filters comprise a plurality of electrodes distributed inside the microchannels. By periodically switching the polarity of the electrodes, microfluidic flows are generated in the microchannels. All inlet flows to the cell apartment are filtered by the immunoisolation of the micro-electric field array filters before entering into the cell apartment. The micro-electric field array filters provide a physical immune protection to the cells cultivated in the cell apartment against the immune system of the host. The microfluidic flows driven by the micro-electric field array accelerate the release of the hormones secreted by the cells cultivated in the cell

### FIELD OF INVENTION

The present invention relates to a micro organism cultivation device, especially to a micro organism cultivation device that provides filtering functions for tiny hazardous particles. The present discloses a structure of the micro organism cultivation device and preparation method thereof.

### BACKGROUND OF INVENTION

According to an estimation made in 1995, the population of diabetics will increase from 135 million in 1995 to 300 million in 2025 worldwide. Among the diabetics, the majority are elder citizens aged over 60. According to IPTR (International Pancreas Transplant Registry), up to December 1997, there were 11,000 SPK's (simultaneous pancreas plus kidney transplants) worldwide. For these people, the survival rate in the first year of the transplant operation is about 94%, in the fifth year is 80%. It has been proved that, if sufficient healthy islet cells are supplied into the body of the sick, diabetes may be well controlled. Although the most effective way to supply healthy islet cells into the body of the sick is the transplantation of islet, such an operation involved high risks. Hyperacute rejections are always reported. In addition to that, supply of healthy pancreases can never satisfy the need of the diabetics. As a result, xenograft islet transplantation, e.g., healthy porcine pancreatic islets, is under development by many institutions of the world.

Experiments on animals proved that the islet cells must contact with the blood of their host closely so that the variation of concentration of blood glucose in the blood may be monitored and insulin may be timingly secreted and transported to the target cells distributed in the whole body. However, when the implanted xenograft islet cells contact with the blood of the host closely, it shall face the attacks from immune reactions. As a result, how to provide the implanted cells with improved defense capability against immune reactions, has become an important task in the field of the xenograft islet transplantation technology.

In the conventional art, encapsulation of islet cells by alginate/poly-L-lysine/alginate (APA encapsulation) is one of the mature means to protect the implanted cells against immune reactions. According to some studies, isolated islet cells of rat as encapsulated by APA encapsulation survived for 80 days in the body of NOD(Non-Obese-Diabetic) mouse hosts. Thereafter, the cells gradually collapsed and dissolved.

Ferrari et al. disclosed a microfabricated immunoisolation membrane in 1998. The microfabricated immunoisolation membrane was tested in an in vitro cell cultivation of isolated islet cells. The experience showed that the membrane is effective in protecting the pancreatic islet cells from the invasion of IgG's (immunoglobulin G). (See T. A. Desai, D. J. Hansford, W. H. Chu, T. Huen and M. Ferrari: “Investigation islet immunoisolation parameters using microfabricated membranes”, Mat. Res. Soc. Sys. Proc., Vol. 530, 1998.)

In 1999 the same research team disclosed further details of the isolation membrane technology. A microfabricated nanometer filter layer is prepared from a sandwiches P⁺ poly silicon/oxide/P⁺ poly silicon sacrificial layer technology. A membrane with high mechanical strength and pores with conformed size are prepared. The size of the pores in the membrane could be as small as 10 nm. (See W. H. Chu, R Chin, T. Huen, M. Ferrari: “Silicon membrane nano filters form sacrificial oxide removal”, J. Microelectromechanical Systems, Vo. 8, No. 1, 1999.)

The vascular bioartificial organ disclosed in U.S. Pat. No. 5,534,025, issued to Moussy, is an improvement to the implantation of pancreatic islet cells. The technology shown in this patent related to implanting islet cells in between the vein and the artery, of the host, such that the cells may contact with the blood of the host closely to enable their biological functions. This patent, however, did not discuss on the details of the immunoisolation between the islet cells and the blood of the host.

The bioartificial pancreas disclosed by Fournier et al. in U.S. Pat. No. 5,855,616 is another novel creation. This patent disclosed a layer of fibers surrounding the vascular structure carrying pancreatic islet cells. The fibers or foam matrix are soaked in a solution containing cellular growth factors before being applied to the vascular structure, to provide small capillary growth and to prevent the blood from clotting. However, whether the speed of growth of the host cells is quick enough to prevent the attack coming from the immune system of the host, is questionable.

In the conventional art, the major task of research is to narrow the cutoff pore size of the immunoisolation membrane. In general cases, the cutoff pore size of a microcapsule may be about 10-30 nm. A microfabricated nano filter layer may have a cutoff pore size of about 10 nm. With such a cutoff size, it is possible to prevent the invasion of particles of 100-200 kDa in molecular weight. A high molecular membrane with a poly-L-lysine layer is able to retard molecules with 60 kDa and above in molecular weight. Such membranes are able to provide immunoisolation effects against representative objects in the immune system, such as lymphocyte or IgG (about 150 kDa).

Unfortunately, it has been shown in the recent studies that attacks to the implanted cells are brought by articles with far smaller sizes. These include cytokine (15-25 kDa) and chemokine (8 kDa). Among them, the interleukin-Iβ involved in the immune system of the CD4 helper T cells will not only cause the cytotoxic aldehyde reactions of the implanted cells but will also actuates the apotosis of the cells. On the other hand, although the molecular weight of the hormone generated by the implanted cells may be small enough to pass the immunoisolation membrane, the hormone could combine with other articles upon its being released. For example, the molecular weight of the insulin is about 6 kDa, upon its being released it is combined with C-peptide as a proinsulin. The volume of the proinsulin is close to, or even greater than that of the cytokine or the chemokine. This created a paradox: volume of particles to be retarded may be smaller that volume of particles to be released. As a result, simply narrowing the pore size of the isolation membrane does not help to establish the immunoisolation system.

It is thus necessary to provide a novel immunoisolation system for implanted cells that is able to provide separate filtering functions to the inlet blood and the outlet blood.

More specifically, it is necessary to provide an immunoisolation system for implanted cells to cut off, and to allow the pass of, articles in inlet blood and outlet blood separately, as follows:

| | | |
|---|---|---|
| Inlet: | WBC, Antibody, Cytokine, Chemokine | cutoff |
| | Glucose, Oxygen, Nutrients | pass |
| Outlet: | Retrovirus, Antigen | cutoff |
| | Insulin, Glucagon | pass |

### OBJECTIVES OF INVENTION

The objective of this invention is to provide a novel micro organism cultivation device that is able to provide separate filtering functions to the inlet blood and the outlet blood.

Another objective of this invention is to provide a micro organism cultivation device that is able to filter hazardous articles from non-hazardous and nutrient articles.

Another objective of this invention is to provide a micro organism cultivation device that provides immunoisolation functions.

Another objective of this invention is to provide a method for the preparation of the above micro organism cultivation devices.

### SUMMARY OF INVENTION

According to the micro organism cultivation device of this invention, an implantable bio-artificial micro device, i.e., a cell apartment, is provided to cultivate cells or tissues. The cells or tissues to be cultivated include that secrete hormones such as islets of Langerhans. At both sides of the cell apartment, provided are microfluidic channels comprising dynamic micro-electric field array filters. The dynamic micro-electric field array filters comprise a plurality of electrodes distributed inside the microchannels. By periodically switching the polarity of the electrodes, microfluidic flows are generated in the microchannels. All inlet flows to the cell apartment are filtered by the immunoisolation of the micro-electric field array filters before entering into the cell apartment. The micro-electric field array filters provide a physical immune protection to the cells cultivated in the cell apartment against the immune system of the host. The microfluidic flows driven by the micro-electric field array accelerate the release of the hormones secreted by the cells cultivated in the cell apartment.

These and other advantages and objectives of this invention may be clearly understood from the detailed description by referring to the following drawings.

### BRIEF DESCRIPTION OF DRAWINGS

FIG. 1 illustrates the plan view of the micro organism cultivation device of this invention.

FIG. 2, a to f, shows the flow chart of the preparation of the micro organism cultivation device of this invention.

### DETAILED DESCRIPTION OF INVENTION

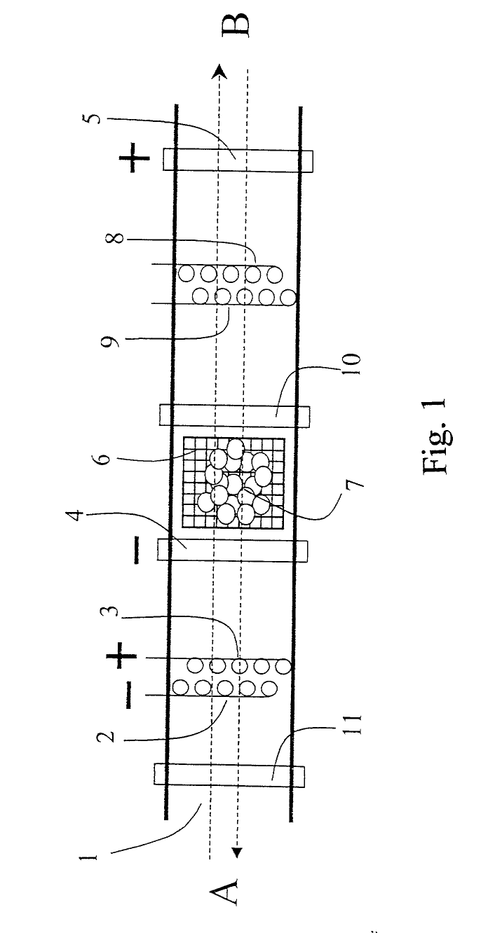

FIG. 1 illustrates the plan view of the micro organism cultivation device of this invention. As shown in this figure, the micro organism cultivation device of this invention comprises a microchannel 1, a cultivation module 6 in said microchannel 1, a first electrode array 2 and 3, and a second electrode array 4 and 5, both positioned beside the cultivation module 6.

The microchannel 1 may be prepared in a substrate (not shown), allowing a microfluid, such as the blood, to pass through. The cultivation module 6 is provided in the microchannel 1 to contain living cells 7 to be cultivated. The fist electrode array 2 and 3 functions as an electro-hydrodynamic pump and an immunoisolation filter for the inlet microfluid simultaneously. The second electrode array 4 and 5, on the other hand, functions as an electro-osmosis pump for the outlet microfluid.

The cultivation module 6 of this invention comprises a microfabricated rigid geometric structure and functions as a cell apartment, such that micro organisms 7 (such as islet cells) may be cultivated inside the cell apartment. In general applications, the surface of the cell apartment 6 may be coated with bio-compatible materials, such as Parylene-C.

The first electrode array comprises a positive electrode array 3 and a negative electrode array 2. Both the positive electrode array 3 and the negative electrode array 2 comprise a plurality of pillar electrodes distributed in the microchannel, in an interlock arrangement. In some other embodiments of this invention, the electrode arrays 2 and 3 are prepared with electrode strips, electrode plates or electrode forks.

When a voltage is applied to the first electrode array 2 and 3, electron flow circles will be formed between the positive electrode array 3 and the negative electrode array 2, through the microfluid therebetween. The electron flow circles will carry the fluid at the circumstance to flow along the direction of the electron flow and an EHD pumping effect is carried out. At the same time, the micro-electric field among the electrodes of the negative electrode array 2 forms a capture net to intercept the IgG, cytokine particles and the chemokine articles that carry negatives. In other words, the electro-hydrodynamic pump provides driving forces to the inlet microfluid and functions as an immunoisolation filter for the inlet microfluid simultaneously.

The second electrode array 4 and 5 functions as the driving force provider for the outlet fluid from the cultivation module 6. The second electrode array 4 and 5 comprises a negative electrode 4 and a positive electrode 5. In this embodiment, both electrodes comprise a metal strip affixed to the wall of the microchannel 1, perpendicularly to the direction of the micro-flow inside the microchannel 1. When a negative voltage is applied to the negative electrode 4 and a positive voltage is applied to the positive electrode 5, opposite charges are formed in the solution near the channel wall, whereby a local electrical gradient is formed. The charges in the microfluid can then be moved under the external applied electric field, in turn, drive the microfluid to flow from electrode 4 to electrode 5. As a result, the second electrode array 4 and 5 functions as an electro-osmosis pump for the microfluid inside the microchannel 1. Due to the electro-osmosis pump 4 and 5, the hormone secreted by the cells 7 (such as islet cells) inside the cell apartment 6 may be transported to outside the cell apartment 6. In this embodiment, the electro-osmosis pump 4 and 5 functions as the major driving force provider for the microfluid in the microchannel 1.

In the embodiment shown in FIG. 1, the micro organism cultivation device comprises two groups of electro-hydrodynamic pumps 2, 3 and 8, 9 and two groups of electro-osmosis pumps 4, 5 and 10, 11. A controller (not shown) may be used to control the application of voltages to these pumps to generate driving forces to the microfluid in the microchannel 1 with different directions, as shown by separate arrows in FIG. 1.

The flow directions of the microfluid in the microchannel 1 may be controlled as shown in the following Table I.

TABLE I

| Electro-hydro- | | | | Electro-osmosis | | | | |
|---|---|---|---|---|---|---|---|---|
| dynamic pump | | | | pump | | | | Flow |
| 2 | 3 | 8 | 9 | 4 | 5 | 10 | 11 | direction |
| − | + | | | − | + | | | A→B |
| | | − | + | | | − | + | B→A |

When the driving mode of the microfluid is from A to B, among electrodes 2 and 3 of the electro-hydrodynamic pump left to the cultivation module 6 is generated a local micro-electric field to function as an immunoisolation for the cultivation module 6. As the micro organisms 7 are positioned between electrodes 4 and 5, released articles secreted by the micro organisms 7, such as insulin, may be easily transported to the microchannel 1 by the electro-osmosis effects of these electrodes 4 and 5. At the same time, no voltage is applied to the other group of electro-hydrodynamic electrodes 8 and 9, whereby no articles will be captured by the electric field generated by electrode array 8 and 9. The article secreted by the micro organisms 7 may be released to the microfluid.

On the other hand, when the driving force is from B to A, as shown in FIG. 1, an immunoisolation is generated at the right side of the cultivation module 6 by electrode array 8 and 9. As the micro organisms 7 are positioned between electrodes 10 and 11, the electro-osmosis pumping force generated by electrodes 10 and 11 drives the microfluid so to transport articles secreted by the micro organisms 7 out of the cultivation module 6. At this time, electrode array 2 and 3 is not supplied a voltage, whereby no articles will be captured by the electric field to be generated. The secreted articles may thus be easily released to the microfluid.

In the application of the micro organism cultivation device of this invention, an external power supply controller (not shown) is used to cyclically switch the flow direction of the microfluid. As a result, all inlet flow of the microfluid into the cultivation module is filtered by the micro-electric field immunoisolation provided by the electro-hydrodynamic electrode array. All outlet flow of the microfluid, on the other hand, is driven by the electro-osmosis pump from either direction.

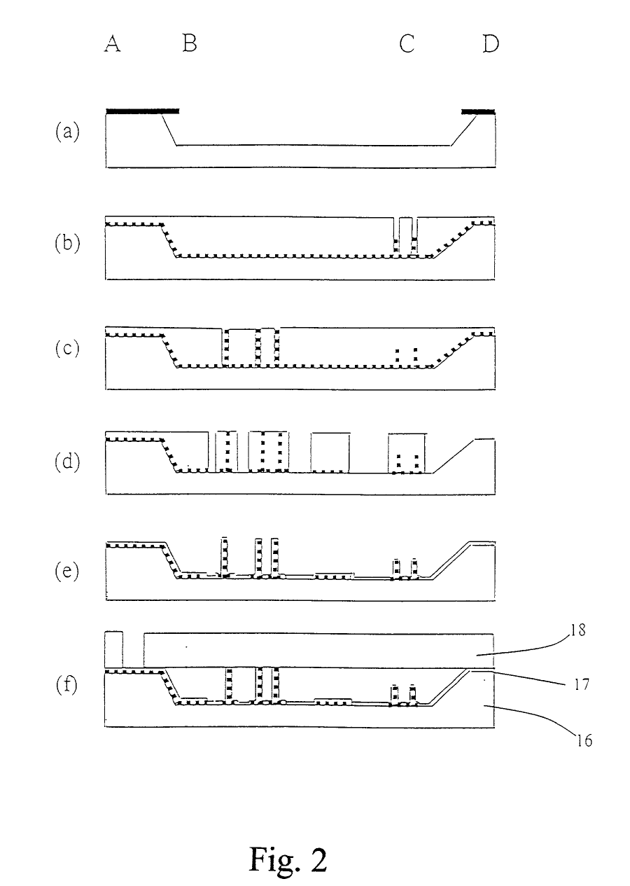

FIG. 2, a through f, shows the flow chart of the preparation of the micro organism cultivation device of this invention.

As shown in this figure, the micro organism cultivation device of this invention may be prepared according to the following steps:

Step a, preparation of chip: At step a, a substrate is prepared and a deep microchannel is formed in the substrate. The substrate is preferably a silicon substrate. A SiN₄ mask layer is formed on the substrate and the substrate is etched in a KOH, THAM (tetramethyl ammonium hydroxide) or EDP (ethylene diamine pyrozine) H₂O solution until a microchannel with necessary depth (e.g., 200-300 nm) is formed.

Step b, preparation of electrodes and cultivation module: At step b, the SiN4 mask layer is removed with a H₃PO₄. A Cr and Au layer is sputtered on the substrate to function as the seed layer of the electrodes. Spin coat a thick photoresist layer. The photoresist layer shall be able to cover the seed layer such that the matrix pattern of the structure of the cultivation module may be prepared with the micro-lithographic technology. Thereafter, electroplate Au to the seed layer to form the cultivation module. The height of the Au layer may be about one third to one half of the depth of the microchannel.

Step c, preparation of electro-hydrodynamic electrodes: The thick photoresist is removed. Spin coat a thick photoresist layer. Again, this photoresis layer shall totally cover the seed layer. The matrix pattern of the electro-hydrodynamic electrodes is prepared with the micro-lithographic technology. Thereafter, Au is electroplated to form the electrodes.

Step d, preparation of electro-osmosis electrodes: The photoresist is removed. Spin coat a thick photoresis layer. This photoresist is required to totally cover the seed layer. The pattern of the electro-osmosis electrodes is prepared with the micro-lithographic technology. The product is subject to etching of the Cr and Au layer to form the electrodes.

Step e, formation of insulation layer: The photoresist is removed. Deposit a paryline high molecular insulation layer 17 with the chemical vapor deposition technology. Such an insulation layer provides the conformal deposition effects to the high-depth pattern of the structure.

Step f, cover: At step f, a glass layer 18 prepared with through holes for lead pads or for the entrance of the micro organisms is anode bonded with the chip prepared in the previous step. A micro organism cultivation device is thus prepared.

### EFFECTS OF INVENTION

In the micro organism cultivation device of this invention, the rigid structure prepared with the semiconductor process provides an uniformed and accurately defined geometric arrangement to cultivate the micro organism. Such arrangements help to improve the affixation of the cultivated micro organisms to the cell apartment and the stability of their biological functions.

In the present invention, the inlet flow and the outlet flow are separately treated. All inlet flows in either direction are subject to the immunoisolation provided by the micro electric field generated by the electro-hydrodynamic electrode arrays and all outlet flows are driven by the electro-osmosis electrodes, each having its respective operation. As a result, different standards are applied to the treatments of the inlet flow and the outlet flow separately. Such a design provides a breakthrough to the paradox of the conventional art.

As in an embodiment of this invention, the microfluidic flow is cyclically shifted in directions. It is thus possible to avoid accumulation of blood corpuscles, protein molecules, antibodies and cell hormones at the micro electric field immunoisolation area. It is also possible to accelerate the supply of nourishments to the cultivated cells, the ventilation of wastes and the release of hormones such as insulin.

In some embodiments of this invention, a bi-directional driving system is used to drive the microfluid. In such a design, it is not necessary to provide two groups of driving force providers. In addition, the driving force provider is not limited to the electro-osmosis pumps as shown in the embodiment of this invention.

In this invention, it is possible to provide a special function in avoiding the adhesion of the blood cell or the protein fibers, when the microfluid is the blood. It is majorly because any inlet blood is at the negative electrodes of the electro-hydrodynamic pump where electrical rejection is generated to avoid the blood cells from being contacted with the electrodes. Although the outlet blood is at the side of the positive electrode of the electro-osmosis pump, the substantial current rejection is strong enough to push away the blood cells.

Although this invention is suited in cultivating cells in blood, it is understood that it is suited in any microfluid. The microfluid is not limited to blood or human blood. The organisms to be cultivated are not limited to animal cells. Other micro organisms such as hepatocytes, endocrine cells, bacteria, tissue . . . etc. may be cultivated in the cultivation device of this invention.

As the present invention has been shown and described with reference to preferred embodiments thereof, those skilled in the art will recognize that the above and other changes may be made therein without departing form the spirit and scope of the invention.

What is claimed is:

1. A micro organism cultivation device comprising:
a microfluidic channel to allow a microfluid to pass through;
a organism cultivation module positioned in said microfluidic channel to contain cells or tissue to be cultivated, allowing said microfluid to pass by said cell or tissue;
a micro electric field generating device positioned in said microfluidic channel and comprising a plurality of positive electrodes and negative electrodes whereby an electronic circuit may be generated to form a micro electric field in said microfluid at an area adjacent to said micro electric field generating device by applying a voltage thereto; and
a microfluid driving device positioned in said microfluidic channel to generate a driving force to drive said microfluid to flow in said microfluidic channel;
characterized in that said micro electric field generating device is positioned at the upstream position of said microfluid relatively to said organism cultivation device.

2. The device according to claim 1 wherein said microelectric field generating device comprises two groups of electrode arrays positioned at both sides to said organism cultivation device respectively.

3. The device according to claim 1 wherein said microfluid driving device comprises two one-directional microfluid drivers positioned in said microfluidic channel, adjacent to both sides of said micro organism cultivation module, respectively.

4. The device according to claim 2 wherein said microfluid driving device comprises two one-directional microfluid drivers positioned in said microfluidic channel, adjacent to both sides of said micro organism cultivation module, respectively.

5. The device according to claim 2 or 4 wherein said microelectric field generation device comprises two micro electric field generating devices, each being supplied with a voltage when positioned at a upstream position of said microfluid.

6. The device according to claim 5, further comprises a control device to cyclically drive to activate a one-directional microfluid driver, together with a micro electric field generating device positioned at an upstream position relative to driving direction of said driver, and another one-directional microfluid driver, together with another micro electric field generating device.

7. The device according to claim 1, 2, 3 or 4 wherein said micro organism cultivation module comprises a rigid microstructure.

8. The device according to claim 7 wherein surface of said micro organism cultivation module is coated with a bio-compatible materials.

9. The device according to claim 1, 2, 3 or 4 wherein said micro electric field generating device comprises at least one array of positive electrode poles and at least one array of negative electrode poles.

10. The device according to claim 1, 2, 3 or 4 wherein said micro electric field generating device comprises at least one array of positive electrode plates and at least one array of negative electrode plates.

11. A method to prepare a micro organism cultivation device, comprising:
preparing a substrate;
forming a microfluidic channel in said substrate;
forming a micro organism cultivation module and a seed layer for at least one group of micro electric field generating electrodes in said microfluidic channel;
forming at least one group of micro electric field generating electrodes in said microfluidic channel; each comprising at least one group of positive electrodes and at least one group of negative electrodes; and
forming at least one group of microfluidic driving electrodes, each comprising at least one positive electrode and one negative electrode.

12. The method according to claim 11 wherein said group of micro electric field generating electrodes comprises two groups of micro electric field generating electrodes, each positioned adjacent to both sides of said micro organism cultivation device, respectively.

13. The method according to claim 11 wherein said group of microfluidic driving electrodes comprising two groups of microfluidic driving electrodes, each positioned adjacent to both sides of said micro organism cultivation device, respectively.

14. The method according to claim 11, further comprising a step of coating to surface of said micro organism cultivation module a bio-compatible material.

15. The method according to claim 14 wherein said bio-compatible material is Parylene-C.

16. The method according to claim 12 wherein said micro electric field generating electrodes comprise electrode poles.

17. The method according to claim 12 wherein said micro electric filed generating electrodes comprise electrode plates.
